(19) United States
(12) Patent Application Publication
(10) Pub. No.: US 2026/0191703 A1
(43) Pub. Date: Jul. 9, 2026

(54) Menstrual Pad in a Dispenser Box

(71) Applicant: Egal Pads Inc., Somerville, MA (US)

(72) Inventor: Thomas E. Devlin, Winchester, MA (US)

(21) Appl. No.: 19/129,463

(22) PCT Filed: Nov. 23, 2022
(86) PCT No.: PCT/US2022/050903

§ 371 Date: May 13, 2025

(51) Int. Cl. A61F 13/551; B65D 83/08; B65D 85/671

(52) U.S. Cl. CPC A61F 13/5514; B65D 83/0805; B65D 83/0894; B65D 85/671; A61F 2013/55195

(57) ABSTRACT

Dispensing systems for bulk distribution of menstrual pads include those having a container with a removable opening for dispensing a stack of pads where the permeable layer of each pads faces away from the opening. The opening can be on the top, for dispensing in the upwards direction like a tissue box, or the container can be mounted on a wall to permit downward dispensing. In some cases, the pads are on continuous strips that are fan-folded or rolled and then dispensed from an opening in the box. In some cases, the opening has a constriction to hold on to the strip to prevent it from falling in.

### FIELD OF THE INVENTION

The present invention relates to menstrual pads in bulk distribution form.

### BACKGROUND

Women often need to carry menstrual pads in case their menstrual cycles unexpectedly begin. These are generally discreet packets that can be carried in a purse. Recent activism has demanded that these pads be made more available in public for free so that people that have periods are not reliant on being ready at all times.

Individual packets often require extra packaging to make them portable and resistant to contamination. Such packets do not pack or stack easily. A bulk delivery format could save on this extra packaging and provide a neater delivery method.

### SUMMARY

The invention is based on the possibility of saving on packaging material and providing orderly dispensing of menstrual pads by providing a distribution unit in which menstrual pads are strategically placed in a box that surrounds and protects them from contamination. A distribution unit of this type could be placed on a table-top or counter like a box of tissues. It could also be inverted and placed in a dispenser, much like a paper towel dispenser or used to hold a continuous strip of such pads.

Individually die-cut menstrual pads with adhesive for attaching to an undergarment can be economically stacked in a box for purchase without the need for individual wrapping of pads if the bottom fluid-impervious layer faced outward toward the opening to the outside.

Alternately, the pads can be fully wrapped and stacked in the box if the goal was only to use a box. The box would generally be a disposable material such as cardboard.

Embodiments of such a box include those having an opening at the top, in which case the box dispenses pads in much the same way that a tissue box dispenses tissues. Also among the embodiments are those that have an opening at the bottom. Such embodiments dispense pads in much the same way that a paper towel dispenser in a public bathroom dispenses paper towels. The latter version described would require a wall-mounted dispenser to expose the bottom face.

An alternative embodiment avoids the need for stacking individual pads. It does so by using a continuous strip of pads that have been fan-folded and placed inside a box. Unlike the preceding embodiment, which dispenses only one pad at a time, this alternative embodiment allows one to pull out at a strip of pads that are still connected to each other. Such an embodiment could also be produced more easily. After all, there would be no need to provide machinery that handles individual pads. This can also be done with fully wrapped strips of pads if desired.

In some embodiments, a box for fan folded strips of pads could have an opening at the top for easy access. Other embodiments feature a lateral opening with a constriction that will hold the remaining pads after removal of one pad.

An alternative embodiment uses a roll of pads as an alternative to a fan-folded strip of pads. Such embodiments feature an opening at a bottom of a box where the roll would rest. The act of pulling out a pad thus tends to urge the roll toward the opening and to unwind it.

In one aspect, the invention features a menstrual-pad dispenser that includes a container and a stack of menstrual pads disposed in the container. Each of the pads includes a permeable layer, an absorbent layer, an impervious layer, and an adhesive layer.

Embodiments include those in which the container has an opening that exposes only the impervious layer of each pad such that permeable layer of each pad faces away from the container.

Other embodiments include those in which the container comprises first and second openings. The first opening receives the menstrual pads and the second opening, which faces away from the permeable layer of each pad, dispenses the menstrual pads.

Embodiments of the foregoing include those in which each of the menstrual pads is wrapped in an impervious material and embodiments in which they are not.

In yet another aspect, the invention features a menstrual-pad dispenser that includes a container and a continuous strip of menstrual pads disposed in the container. Each pad has a permeable layer, an absorbent layer, an impervious layer, and an adhesive layer. The container includes an opening for dispensing the menstrual pads.

Among these embodiments are those in which the continuous strip is a fan-folded strip and those in which it is a rolled strip.

Further embodiments include those in which the continuous strip is wrapped in an impervious material and those in which it is not.

These and other features of the invention will be apparent from the following detailed description and the accompanying figures, in which:

### DESCRIPTION OF DRAWINGS

FIG. 1A is a representation of a menstrual pad showing an absorbing region and a sealing region that surrounds the absorbing region to keep fluid from leaking out of it;

FIG. 1B is a variation of the menstrual pad of FIG. 1A showing wings that are used to wrap around an undergarment for more secure placement;

FIG. 1C is a cross section of the menstrual pad of FIG. 1A showing the functional layers and regions, with thicknesses having been exaggerated for clarity of illustration;

FIG. 2A shows the pad of FIG. 1A with a wrapper, which serves as a release paper or is used in conjunction with a release paper, that is about to be folded over;

FIG. 2B is representation of the pad and wrapper in FIG. 2A after having been folded over and sealed from contamination;

FIG. 3A is depiction of a dispenser before assembly showing a stack of pads and a surrounding protective box that is to receive the stack of pads;

FIG. 3B is a representation of the dispenser of FIG. 3A after having been assembled and sealed and after the top has been removed to allow dispensing of a pad in an upward direction;

FIG. 3C is a transparent view of the dispenser shown in FIG. 3A after it has been assembled and sealed and after a portion of a bottom face thereof has been removed so that a stack of pads urged against the bottom face by gravity can be dispensed in a downward direction;

FIG. 4A depicts a system that allows dispensing of pads in the downward direction before assembly comprising the dispenser of FIG. 3A and an open dispenser mounted to a vertical surface;

FIG. 4B depicts a system of FIG. 4A after assembly and ready to dispense pads;

FIG. 5A is depiction of a continuous strip of the pads of FIG. 1A with a perforation between each pad to ease separation;

FIG. 5B is depiction of a continuous strip of the pads of FIG. 2B with a perforation between each pad to ease separation;

FIG. 6A is a transparent view of an assembled box of pads in which one of the continuous strips shown in FIG. 5A and FIG. 5B has been fan-folded to form a stack;

FIG. 6B is view of the box of pads of FIG. 6A with the package having been opened and pads having been pulled through the opening thus formed;

FIG. 6C is view of the box shown in FIG. 6B but with a constricted opening that engages the strip so as to make the next pad easy to grab and to prevent the next pad from falling back inside the box;

FIG. 7A is a transparent view of an embodiment similar to that shown in FIG. 6A but with the strip of pads shown in FIG. 5A or 5B having been wound into a roll; and

FIG. 7B is view of the embodiment shown in FIG. 7B with the package having been opened and with a portion of the strip therein having been pulled through the opening thus formed to present the next pad.

### DETAILED DESCRIPTION

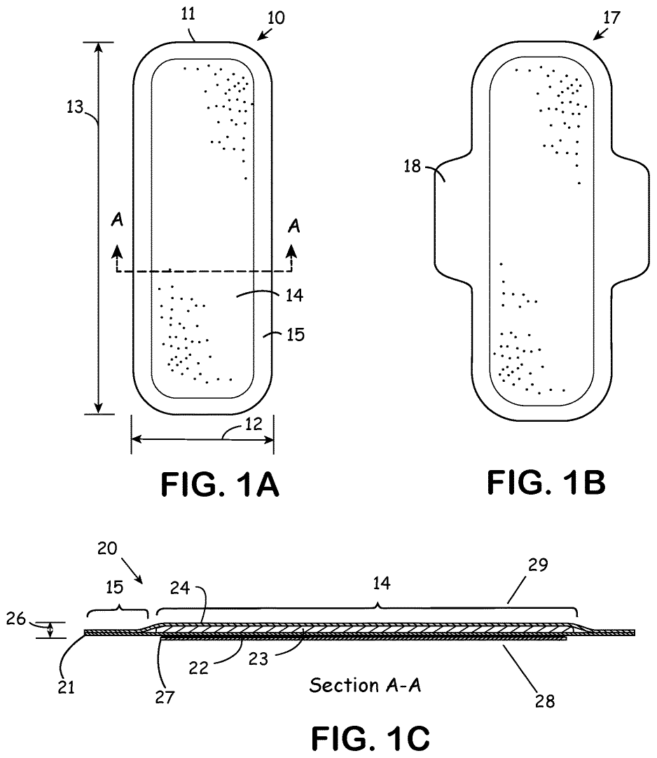

Referring to FIG. 1A, a menstrual pad 10 includes an absorbing region 14 surrounded by a sealing region 15. The absorbing region 14 absorbs and retains fluids. The sealing region 15 ensures that those fluids remain in the absorbing region 14. In a typical embodiment, the pad 10 has short edge 12 of between thirty millimeters and sixty millimeters and a long edge 13 of between 140 millimeters and 320 millimeters.

In the embodiment shown in FIG. 1A, the sealing region 15 has a die-cut shape 11 without wings. In the alternative embodiment, shown in FIG. 1B, the sealing region 15 has a die cut shape 11 that forms extended wing regions 18. These wing regions 18 are useful for wrapping around an undergarment and thereby providing further protection from fluids.

The cross-section along “A-A” in FIG. 1a can be seen in FIG. 1c.

Referring now to FIG. 1C, the menstrual pad 10 include a backing layer 21 between a pressure-sensitive adhesive layer 22 and a fluid-absorbing layer 23. The adhesive layer 22 is located where it is best suited for attaching the pad 10 to an undergarment.

The backing layer 21 comprises a fluid-impervious material. The fluid-absorbing layer 23 and a permeable layer 24 together form the absorbing region 14.

The permeable layer 24 extends beyond the fluid-absorbing layer 23 and into a sealing region 15. At the sealing region 15, the permeable layer 24 is welded to the backing layer 21, thus forming a seal that is impervious to fluid.

An optional release layer 27 covers the adhesive layer 22. In some embodiments, the release layer's area is only large enough to cover the adhesive that is on the adhesive layer 27. However, in some embodiments, the adhesive layer 27 is entirely covered with adhesive, which, as a result, covers the entire area of the pad 10.

The pad 10 thus has two opposed faces: a protective face 28 and an absorbent face 29. When the pad 10 is in use, the protective face 28 does not contact the body. The absorbent face 29, however, is in intimate contact with the body. As a result, the dispensers described herein are configured so that only the protective face 28 is exposed to the environment.

The backing layer 21 comprises a fluid-impervious material. Among the materials suitable for use in the backing layer 21 is a low-density polyethylene film.

The fluid-absorbing layer 23 comprises an absorbent medium. A suitable medium is a fabric, examples of which include a non-woven cotton fabric. Examples of absorbent media include those made of cotton and those made of cellulose.

Other examples of a fluid-absorbing layer 23 include a laminate or assembly that comprises a super-absorbent polymer. A super-absorbent polymer is particularly useful since a fluid-absorbing layer 23 made of such a polymer could absorb three-hundred times its weight in water and thirty to sixty times its volume of water.

In some manufacturing practices, the super-absorbent polymer is sprayed on as a powder during a continuous operation. This results in extremely thin pads that can absorb as much fluid as thicker pads that use cotton or cellulose. The pad's sealing region 15, which in some embodiments is only 0.1 millimeters thick, is even thinner than its absorbing region 14. The thinness of the pads 10 allows many more pads to be put on a roll or stacked within a particular volume. This results in significant cost savings and efficient use of space. In a typical embodiment, the pad's overall thickness 26 is between half a millimeter and two millimeters.

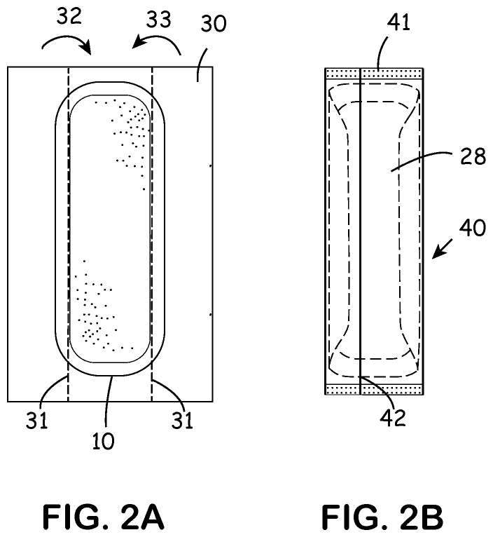

FIG. 2A shows a pad 10 on a piece of fluid-impervious material 30 just before being wrapped. The wrapping process includes folding the pad 10 along fold lines 31 to form a first fold 32 and a second fold 33 that overlaps the first fold.

FIG. 2B shows the result of the folding operation of FIG. 2A followed by applying adhesive 42 to hold the overlapping ends together. The resulting pad 10 has now been sealed from contamination 40 that is incident from two of the three possible directions. A subsequent sealing step that relies on an adhesive or embossing process seals the ends and thus protects the pad 10 from contamination 31 incident from the remaining direction. In this embodiment, the protective face 28 has been made to extend over the entire outer surface of the pad 10.

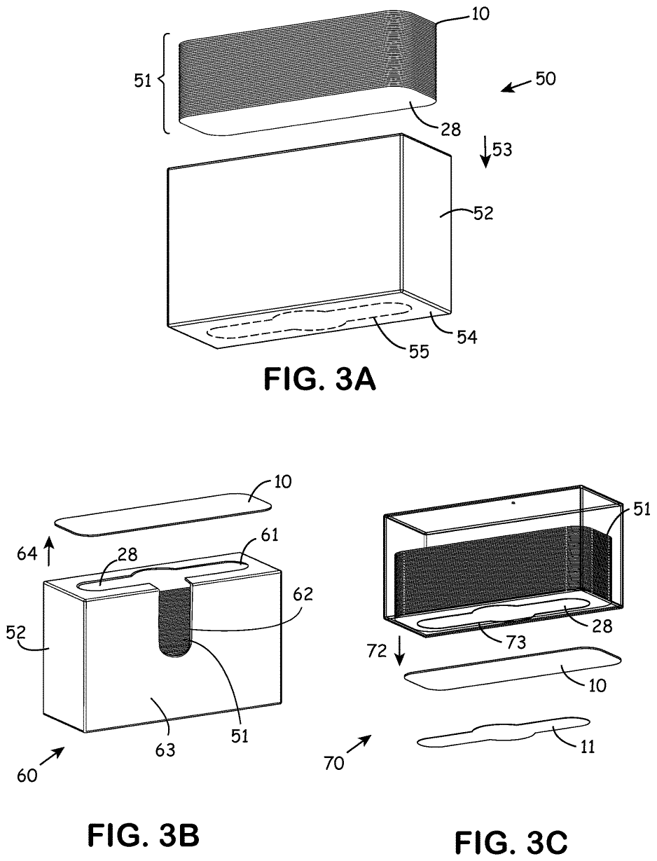

FIG. 3A shows a dispenser assembly 50 that seals a stack 51 of pads 10 therein. The box 52, which is typically cardboard for easy disposal, comprises folded panels that have been glued together. Among the panels is an opening panel 54 having a weakened contour 55 that enables one to form an opening on the opening panel 54 to permit access to the stack 51. In a preferred embodiment, the weakened contour 55 is formed by a perforation.

Each pad's protective face 28 has a normal vector 53 that is directed toward the opening panel 54. As a result, the protective face 28 bears the brunt of being exposed directly to the environment. The more vulnerable absorbent face 29 faces away from the opening panel 54 and thus suffers only reduced contamination compared to the protective face 28.

FIGS. 3B and 3C show dispensing assemblies 60, 70 similar to that shown in FIG. 3A in which the normal vector 53 is directed opposite the gravity vector and in which it is directed in the same direction as the gravity vector, respectively. These will be referred to herein as “upward” and “downward,” respectively.

In the dispenser assembly 60 of FIG. 3B, the box 50 is oriented to dispense pads 10 in an upward direction 64. As was the case in the embodiment shown in FIG. 3A, the protective face 28 of each pad 10 is oriented to face an opening 61. This ensures that the absorbent face 29 faces a direction that minimizes contamination thereof.

Since gravity does not push the stack 51 upwards, it is useful for the upwardly-facing opening 61 to have a lateral extension 62 that extends downward along a lateral face 63 of the box 50. This permits access to the pads 10 even as the supply of pads 10 on the stack 51 diminishes.

In the alternative dispenser assembly 70 shown in FIG. 3C, the box 50 is oriented to dispense pads 10 in a direction 72 through a dispensing opening 73. As was the case in the embodiment shown in FIG. 3A, the protective face 28 of each pad 10 is oriented to face a dispensing opening 61, thereby ensuring that the absorbent face 29 faces a direction that minimizes contamination thereof.

An advantage of dispensing pads 10 in the downward direction 72 is the availability of gravity for ensuring that the stack of pads 51 is always aligned with a dispensing opening 73 for easy access. A corresponding disadvantage is the need for a suspension system to enable access to the dispensing opening 73.

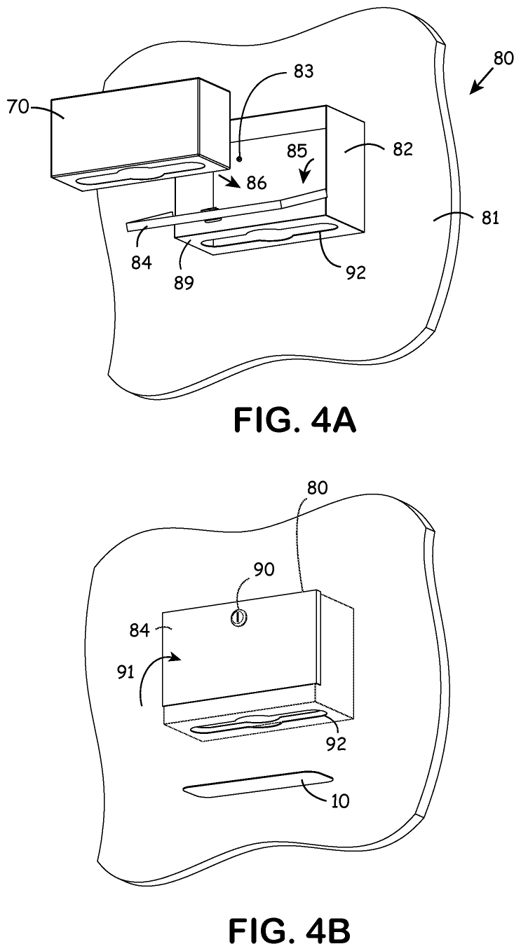

FIG. 4A shows a suspension system 80 for suspending the dispenser assembly 70 from a vertical surface 81. The suspension system 80 includes a wall-mounted container 82 that is mounted to the vertical surface 81 by a mounting screw 83. A bottom face 89 of the wall-mounted container 82 features an aperture 92 that aligns with the dispensing opening 73 of the dispenser assembly 70. A door 84 swings open along an opening direction 85 to allow the dispenser assembly to move in an installation direction 86 into the container 82.

FIG. 4B shows the suspension system 80 ready for use. The door 84 has been swung shut along a closing direction 91 and an optional lock 90 locks the door 84 into place. In this position, it is a simple matter for one to pull a pad 10 out through the aperture 92.

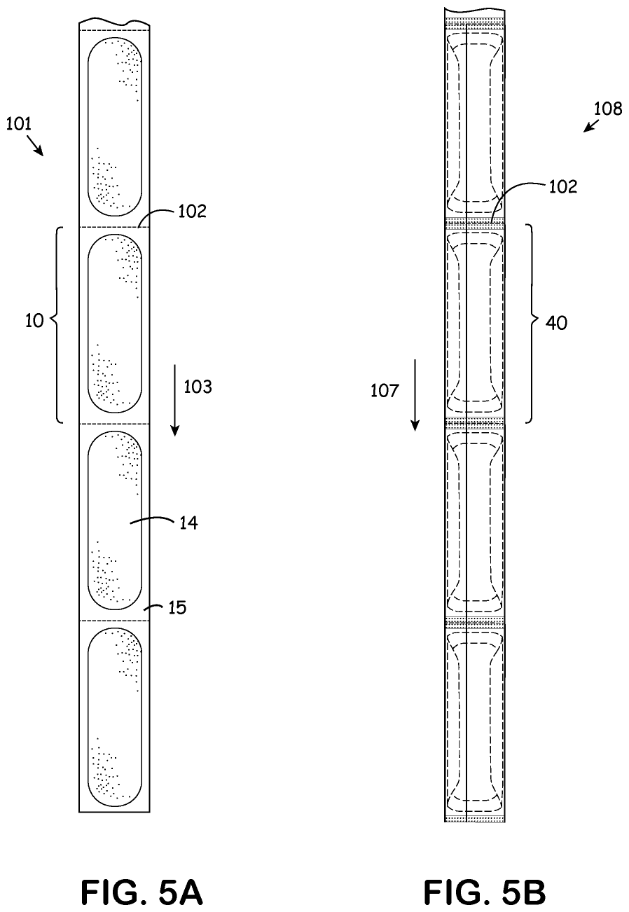

Referring to FIG. 5A, a portion of a strip 101 of unwrapped pads consists of a series 103 of pads 10, each of which is separated from neighboring pads by a perforation 102. The perforation 102 promotes the ability to separate a pad 10 from the strip 101.

FIG. 5B shows a strip 108 having a series 107 of pads 10 similar to that shown in FIG. 5A but with the strip 108 having been wrapped in an impervious material to protect the pads 10 from contamination.

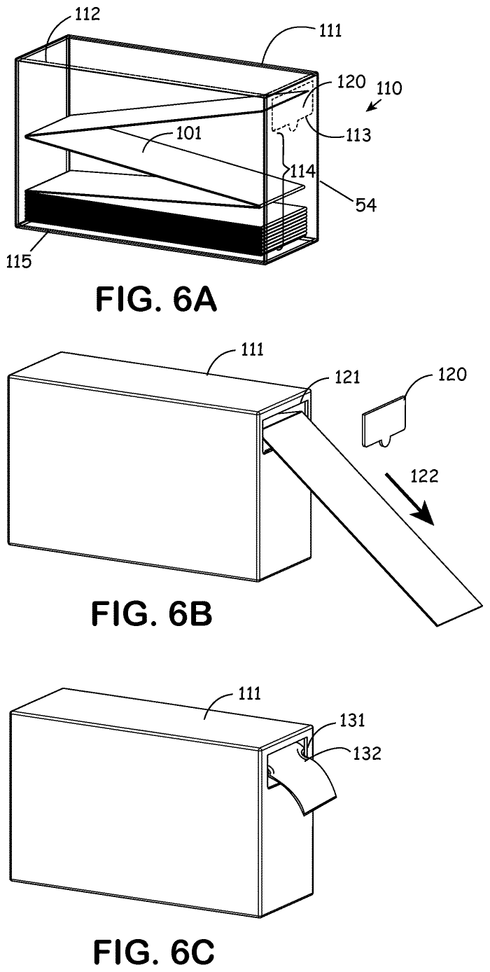

FIG. 6A shows a dispenser 110 for dispensing a strip 101, 108 along the lines of those shown in FIGS. 5A, 5B.

The illustrated dispenser 110 features a box 111 having a top panel 112, a bottom panel 115 opposite the top panel 115, and an opening panel 54 that extends between the top and bottom panels 112, 115. In a preferred embodiment, the box 111 is made of a disposable material, such as cardboard, to permit easy disposal when the pads 10 have all been dispensed.

At an end closest to the top panel 112, the opening panel 54 has a weakened region 113 that is intended to serve as an opening for dispensing the pads 10, for example by tearing at perforations or by opening a glued panel 120.

The strip 101, 108 is fan-folded to define plural folds 114 and then placed in the box 111 so that it rests on the bottom panel 115. An end of the strip 101, 108 is positioned near the weakened region 113 to be available when the box is opened for dispensing.

FIG. 6B shows the box 111 of FIG. 6A after having removed the glued panel 120 to create an opening 121. In this state, it is possible to pull the fan-folded strip 101, 108 in a dispensing direction 122. Since the pads 10 are on a continuous strip, doing so pulls trailing pads to the opening 121.

A difficulty that can arise is that the end of the strip 101, 108 will inadvertently fall out of the opening 121 and back into the box 111, thus becoming inaccessible. To avoid this, the embodiment shown in FIG. 6C includes a constricted opening 131 having a constriction 132 to engage the strip 101, 108, thereby holding it in place so that it does not fall back into the box 111.

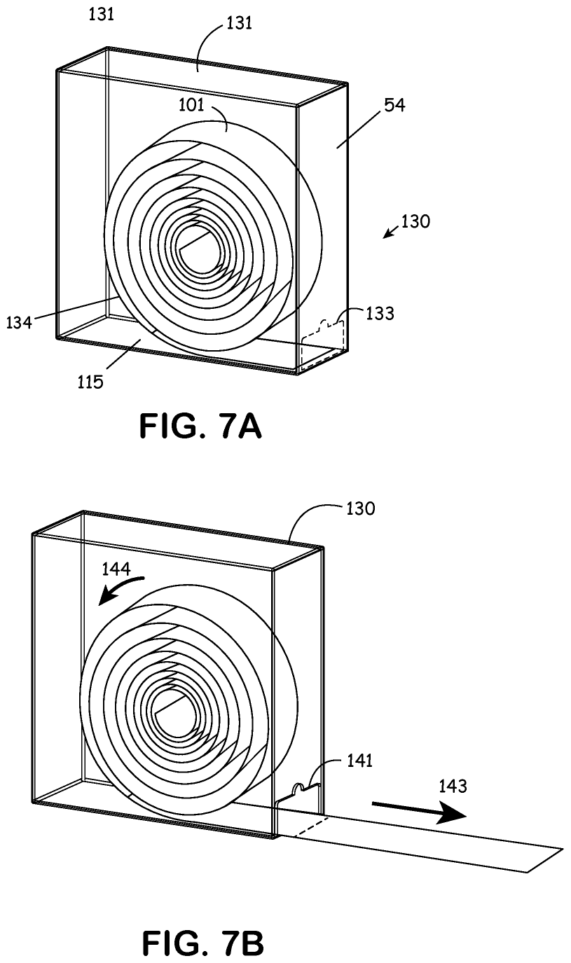

Referring now to FIGS. 7A and 7B, an alternative dispenser 130 accommodates a strip 101, 108 of pads 10 that have been rolled into a roll 134.

Referring to FIG. 7A, the dispenser 130 includes a dispenser box 131 having a top panel 112, a bottom panel 115 and an opening panel 54 that extends between the top and bottom panels 112, 115. The box 131 is generally made of a disposable material, such as cardboard, to permit easy disposal when the pads 10 have all been dispensed.

At an end closest to the bottom panel 116, the opening panel 54 has a weakened region 133 that is intended to serve as an opening for dispensing the pads 10, for example by tearing at perforations or by opening a glued panel.

A roll 134 that is formed by rolling the strip 101, 108 is then placed in the dispenser box 131 so that it rests on the bottom panel 115. An end of the rolled strip 101, 108 is positioned near the weakened region 133 to be available when the box is opened for dispensing.

Referring to FIG. 7B, removing the weakened region 133 creates a dispensing opening 141. Pulling the strip 101, 108 of pads 130 along a dispensing direction 143 causes the roll 134 to rotate in a rotation direction 144 as it unwinds. This ensures that a pad 10 will be available at the opening 141 for the next person who needs it.

The details of an economical manufacturing method of these dispensers such as materials, bonding, adhesives, methods for attachment, perforating, and die-cutting have been left out for clarity, and it is assumed that those skilled in the art of packaging manufacturing should be able to produce the dispenser described.

It is to be understood that the foregoing description is intended to illustrate and not to limit the scope of the invention, which is defined by the scope of the appended claims. Other embodiments are within the scope of the following claims.

1. A manufacture comprising a container and a stack of menstrual pads disposed in said container, each of said pads comprising a permeable layer, and absorbent layer, an impervious layer, and an adhesive layer, wherein said container comprises an opening that is oriented to expose only said impervious layer of each pad, and wherein said permeable layer of each pad faces away from said container.

2. The manufacture of claim 2 where each menstrual pad is wrapped in an impervious material.

3. A manufacture comprising a container and a stack of menstrual pads, each pad having a permeable layer, and absorbent layer, an impervious layer, and an adhesive layer, wherein said container comprises first and second openings, said first opening for receiving said menstrual pads and said second opening for dispensing said menstrual pads, wherein said second opening faces away from said permeable layer of each pad.

4. The manufacture of claim 3 where each of said menstrual pads is wrapped in an impervious material.

5. A manufacture comprising a container and a fan-folded stack of a continuous strip of menstrual pads disposed in said container, each of said pads having a permeable layer, and absorbent layer, an impervious layer, and an adhesive layer, said container comprising an opening for dispensing for dispensing said menstrual pads.

6. The manufacture of claim 5 wherein said continuous strip of menstrual pads is wrapped in an impervious material.

7. A manufacture comprising a container and a rolled continuous strip of menstrual pads, each of said pads having a permeable layer, and absorbent layer, an impervious layer, and an adhesive layer, said rolled continuous strip having been disposed in said container, said container comprising an opening in the container for dispensing.

8. The manufacture of claim 7 where said continuous strip of menstrual pads is wrapped in an impervious material.
